(19) United States
(12) Patent Application Publication
(10) Pub. No.: US 2013/0281888 A1
(43) Pub. Date: Oct. 24, 2013

(54) TASK-ASSOCIATED MOTION ANALYSIS

(71) Applicants: Robert Bender, Ottawa (CA); Trevor Jones, Ancaster (CA); Wendy Porter, Mississauga (CA)

(72) Inventors: Robert Bender, Ottawa (CA); Trevor Jones, Ancaster (CA); Wendy Porter, Mississauga (CA)

(21) Appl. No.: 13/651,849

(22) Filed: Oct. 15, 2012

Related U.S. Application Data
(60) Provisional application No. 61/547,524, filed on Oct. 14, 2011.

(51) Int. Cl. A61B 5/11; A61B 5/00

(52) U.S. Cl. CPC A61B 5/1116; A61B 5/0002; A61B 5/72
USPC 600/595

(57) ABSTRACT

Systems and methods for task-associated motion analysis are provided. In the task-associated motion analysis system, a protocol including one or more motion-based tasks is performed by a patient. The patient's performance is recorded using one or more sensors and the patient's data is compared to normative data to assess musculoskeletal performance for the purposes of diagnosis and administration of therapy or training recommendations.

### CROSS-REFERENCE TO RELATED APPLICATIONS

The present application claims the benefit of U.S. Provisional Application No. 61/547,524, filed Oct. 14, 2011, entitled “Task-Associated Motion Analysis”.

### BACKGROUND OF THE INVENTION

The present invention generally relates to assessing musculoskeletal performance. More particularly, the present invention relates to a assessing musculoskeletal performance for the purposes of diagnosis and administration of therapy or training

A general problem in physical training and therapy is that of measurement and analysis of performance. Feedback of performance information is a potentially very useful part of training.

Task performance typically involves multiple physical components which are required to be coordinated. For example, performance of a task such as getting out of a chair or walking requires that a large number of sub-tasks be carried out at a neurological and musculoskeletal level. The end result is typically not a unique event sequence. The event sequent may differ by individual, health status and other circumstances. Daily living, sporting activities, and recovery of function after injury or other adverse circumstance all depend on the ability to perform specific physical tasks. Physical training, rehabilitation and performance measurement all require information about motion tasks.

### BRIEF SUMMARY OF THE INVENTION

One or more of the embodiments of the present invention provide methods and systems of assessing human musculoskeletal performance for the purposes of diagnosis and administration of therapy or training. In the present task-associated motion analysis system, a protocol including one or more motion-based tasks is performed by a patient. The patient's performance is recorded using one or more sensors and the patient's data is compared to normative data to identify decreased performance. When the patient's performance departs from the normative data, recommendations for improvement may be made.

### BRIEF DESCRIPTION OF THE DRAWINGS

FIG. 1 illustrates a task-associated motion analysis system according to an embodiment of the present invention.

FIG. 2 illustrates the rectified COP velocity and the actual COP velocity.

FIG. 3 illustrates an alternative embodiment of the task-associated motion analysis system.

FIG. 4 illustrates an additional alternative embodiment of the task-associated motion analysis system.

FIG. 5 illustrates another alternative embodiment of the task-associated motion analysis system.

FIG. 6 shows a graph of the total force (Ftotal) observed using a force plate for a 53 year old female subject performing a stand-to-sit task.

FIG. 7 shows graphs of the Force top left (FTL), Force top right (FTR), Force bottom left (FBL), and Force bottom right (FBR) for the same subject as in FIG. 6.

FIG. 8 shows a graph of the total force (Ftotal) observed using a force plate for a 86 year old female subject performing a stand-to-sit task.

FIG. 9 shows graphs of the Force top left (FTL), Force top right (FTR), Force bottom left (FBL), and Force bottom right (FBR) for the same subject as in FIG. 8.

FIG. 10 shows a graph of the total force (Ftotal) observed using a force plate for a 53 year old female subject performing a sit-to-stand task.

FIG. 11 shows a graph of the total force (Ftotal) observed using a force plate for a 86 year old female subject performing the same sit-to-stand task as in FIG. 10.

FIG. 12 illustrates multiple performances of the sit-to-stand task by a middle-aged, fit male.

FIG. 13 shows graph of the total force (Ftotal) observed using a force plate for a 53 year old female subject performing a sit-to-stand task.

FIG. 14 shows graphs of the total force (Ftotal) observed using a force plate for a 53 year old female subject, 76 year old female subject, and 86 year old female subject performing a sit-to-stand task.

FIG. 15 shows graph of the total force (Ftotal) observed using a force plate for the middle-aged male subject performing a sit-to-stand task.

FIG. 16 shows graphs of the Force top left (FTL), Force top right (FTR), Force bottom left (FBL), and Force bottom right (FBR) for the same subject as in FIG. 15.

FIG. 17 shows graphs of the center of gravity in the x direction and the center of gravity in the y direction for the same subject as in FIG. 15.

FIG. 18 illustrates a force-time graph showing a subject establishing and maintaining a one-legged stance.

### DETAILED DESCRIPTION OF THE INVENTION

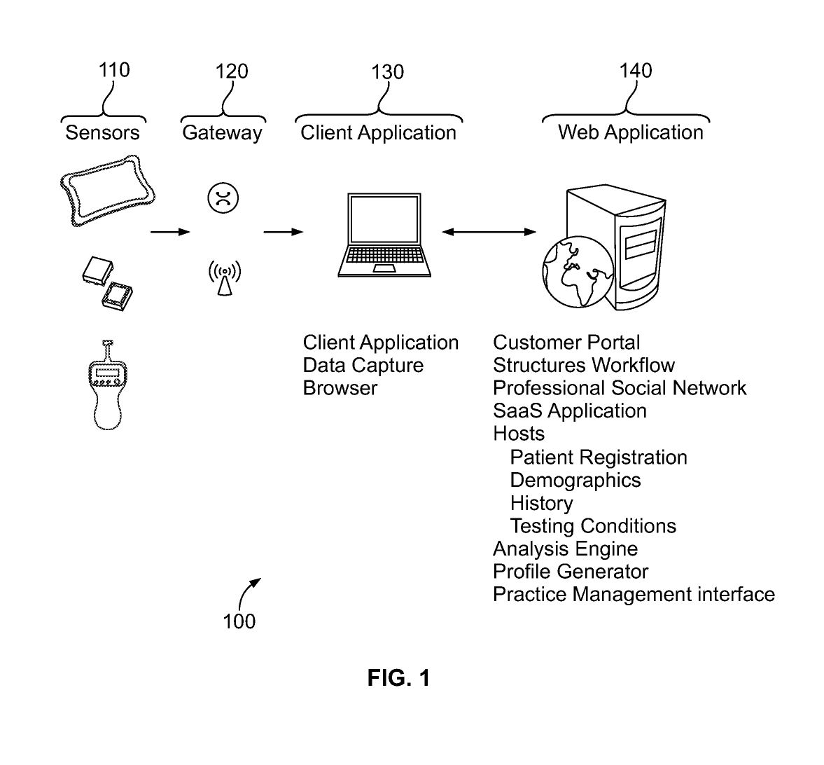

FIG. 1 illustrates a task-associated motion analysis system 100 according to an embodiment of the present invention. The task-associated motion analysis system 100 includes one or more sensors 110, a gateway 120, a client application 130, and a web application 140.

As further described below, in one embodiment a client or patient is positioned so that the sensor 110 is able to sense and record the motion of the patient. The patient is then instructed to perform a predetermined motion task, such as a sit-to-stand task that requires the patient to stand up from a seated position. The sensor 110 records the patient's motion during performance of the task. The data recorded by the sensor 110 may then be passed to the gateway 120 which relays the data to the client application 130.

At the client application 130, the sensor data may be captured and may also be displayed to an operator, preferably through a browser-driven interface installed as part of the client application 130. Additionally, the sensor data may then be passed from the client application 130 to the web application 140.

As further described below, the web application 140 is preferably located at a centralized location and receives information from numerous client applications. As further described below, the web application 140 preferably includes a customer portal, structures workflow, includes a professional social network, includes a Software-As-A-Service application, hosts databases for patient registration, demographics, history, and testing conditions, includes an analysis engine, includes a profile generator, and includes a practice management interface.

Additionally, one or more embodiments provide a system and method of use which may provide assessment of task-related and general motion for use in training, assessment and therapeutic applications such as (but not limited) to athletic performance, physical therapy and rehabilitation. In one embodiment, a subject (S) (patient) interacts with a dynamic measurement device or devices (D_{1 . . . n}). Such measurement devices may include but are not restricted to force plates, accelerometers, inertial measurement units, video capture systems and other sensor systems which may provide data on position, force, rate of motion or other physical results of musculoskeletal action. In general such systems are preferably capable of acquiring data at rates equal to or greater than the repetition rate of the task being measured.

Without limiting generality, further descriptions may refer to force measurement embodiments but dynamic data on either force or position or rate of motion may be used in subsequent analytical steps. Specific embodiments may use low-cost robust consumer products as sensors and computers networked through the internet. A practical consequence of this is portability and ease-of-use.

Additionally, a dynamic measurement device which may explicitly combine several sensor and modalities may yield a time-series of data that corresponds to performance of a defined task or set of tasks (exercise or motion protocol).

This task-associated data is captured in a file by use of an interface device such as client application 130 that also may control the sensor device within a local feedback loop. The data file is annotated (tagged) as to the subject and/or patient identity and task performance particulars (start time, end time and general description) with information provided by the subject through an interface such as client application 130 or by a therapist or operator through the interface. The client application 130 may be a terminal, laptop or mobile device such as a tablet or smartphone. Additionally, the same interface may be used by both subject and operator but in general these inputs are preferably through separate devices linked to the network.

With respect to FIG. 1, the apparatus and its method of use are broadly described. The description is intended to illustrate the flow and management of data within a network. Internal traffic of signals for internal error correction and security (encryption) are not shown, but are preferably present. For example, the communication of the data from the sensors to the gateway, from the gateway to the client application and/or from the client application to the web application may be encrypted—as may be the communication from the web application to the client application.

Similarly, it is understood that linkages between elements of the apparatus may be hard-wired or wireless and that such elements may be co-located or distributed geographically. The network aspects of the apparatus are a preferred aspect of its functioning. It is common practice in networks to distribute ‘intelligence’ and storage elements in order to optimize performance and such elements as would commonly form part of a network are included. Accordingly, a network embodiment may have several forms and include additional data storage or relay elements.

The specific task (or set of tasks) is part of a task library data base which provides the task-related Protocol to which the data is referenced. Protocols may include simple or multiple tasks and constrain performance as to time and expected range of performance. Therefore they may be used to probe aspects of performance of a complex biomechanical system without explicit knowledge of subsystem functioning. In another embodiment of the invention, tasks within Protocols may be modified or perturbed so as to elicit performance changes relevant to the functioning of specific system components. A Protocol is preferably externally defined and not modified by the system except as to duration and/or number of repetitions.

In a practical setting, the measurement and analysis system which is the subject of FIG. 1 is used by an operator or a therapist working with a subject/patient according to FIG. 1. That is, once a test Protocol is selected and initiated (typically bt the operator), the system prompts (or specifies) the specified task(s) and then through use of an appropriate set of sensors task-related performance data is acquired through the gateway 120 which is recorded and transmitted for analysis, feedback and reporting. Optionally, the data acquisition may be directly linked to a display interface to provide immediate confirmation of measurement and performance feedback.

The client application 130 includes an interface device which may have a display and data entry capability so that the user (therapist or subject) may enter relevant annotation and view data as confirmation of task performance.

The data acquired in task-measurement is then transmitted by a hard or wireless link to a server including the web application 140 which hosts an analysis module, and one or more data bases where, using standard methods of mathematical analysis (signal extraction, averaging and statistical processing), relevant performance information may be extracted as further described below.

The server may also link to cloud-based or other servers as is common in data processing networks and thus may access additional data such as patient medical records, demographic or sports performance stored in independent repositories of such data.

A feature of an embodiment is that data from individual subjects and tests is analyzed on-line with data accumulated from other test subjects and tests thus enabling quantitative comparisons of performance; such comparisons may be to normative populations or over time within an individual's performance history.

In addition, the ability to quantify performance of specific tasks allows feedback and task modification as further described below in such ways are specifically and uniquely useful for assessment and training.

For illustrative purposes and not restricting generality, an example of a Protocol to assess postural stability and its use is now described. The Protocol starts with the recognition that low back pain (LBP) sufferers exhibit poorer postural instability than healthy controls in the majority of studies investigating postural instability using center of pressure (COP) analysis. For example, non-specific low back pain sufferers have shown to exhibit one or more of: a greater COP sway area; a higher COP sway velocity; and increased COP mean displacement, particularly in the anterior-posterior direction.

Particularly, COP mean velocity has been shown to be both reliable and discriminative for non-specific low back pain. Further, COP mean velocity has shown to have a linear relationship to pain intensity in non-specific low back pain. This relationship was maintained when pain intensity improves with manual therapy and rehabilitation. Additionally, in the majority of cases, as self-rated pain increases, COP mean velocity increases and as self-rated pain decreases, COP mean velocity decreases. Also, COP sway velocity and 90% COP circle diameter increased linearly with increasing perceived pain intensity in non-specific LBP sufferers. Consequently, postural sway may be used as an objective clinical monitoring tool for patients under treatment or rehabilitation for non-specific low back pain.

One exemplary Protocol for using task-based motion analysis to diagnose, identify, typify, and/or evaluate LBP is now described. First, a subject is instructed to stand and hold position on a balance board (for example, a Nintendo Wii balance board) and measurements of COP are taken during a 3 successive 2 minute intervals. Data from the balance board permits calculation of COP and COP sway velocity, which is then calculated. This data is then compared to a demographically relevant sample in a database which has been previously populated and may be compared to longitudinal (time series) data on the subject in order to assess progression or treatment of non-specific low back pain.

Preferred embodiments of the invention include those in which the data is analyzed with explicit or implicit reference to similar data within a data repository. Methods of data analysis include (without limitation) signal averaging and integration, statistical cluster analysis, frequency spectrum analysis, etc. Pre-processing of signal streams is generally preferred for interpretation and feedback; raw force and rate data from sensor systems is not typically directly interpretable although positional data may be useful as it is comparable to point data currently measured.

An emergent property of measurement data acquired with the present system is the possibility of signal integration and improvement in signal-to-noise by repeated performance and measurement of the same task. Accurate quantitation is preferable for such signal-to-noise improvement and is not available through manual methods due to lack of operator consistency. This has qualitative and unexpected consequences in relation to applications such as rehabilitation where dynamic perturbation of performance may be used to probe the interaction between cognitive and motor impairment. Established standard tasks may be used for both assessment and training.

Efficacy of (re)training for remediation of performance deficits resulting from injuries such as stroke, trauma or other disease may thus be assessed based on the quality of information transfer as measured by dynamic perturbation of performance. Specifically by use of perturbation methods it is possible to separate control noise from performance noise in relation to repetitions of a specific task.

Physical therapy, rehabilitation and physical training are processes in which there is a common methodology which begins with an assessment of initial state or skill, following which there are cycles of performance rehearsal and measurement of change (improvement) as determined by the ‘distance’ from the initial state as assessed along a projected trajectory to a desired (measurable) state. An illustration of this is ‘range of motion’ where a single function may sufficiently characterize performance and improvement. In general, task performance is characterized by multiple measured variables and the distance is defined in an n-space.

In many settings there are complex interactions between the constraints of the physical system (strength, joint mobility and proprioceptive feedback), cognition (visual, sensory inputs and voluntary control) and the effects of repetition which alters both physical and cognitive responses and responsiveness.

It is a desirable (but not easily met) requirement that assessment steps in this process be quantitative and repeatable. Due to the complexity of the systems involved there is often a high subjective factor in such assessments. This may be addressed by measurement strategies which allow easy repetition and thereby improvement of data quality through integration and ‘averaging’ strategies.

Similarly, as cycles of performance rehearsal are undertaken, specific sub tasks may be isolated and both the overall and sub-task performance may be measured (and tracked) with greater facility and accuracy. A common problem of such schema is that of “inappropriate” training—examples of which are often seen in physical training due to the voluntary nature of these tasks and the precise definition of desired end results. “Re-training” generally involves an “unlearning” or corrective prescription as a first step.

Training environments exhibit a dose-response function that is non-linear with respect to the number of cycles and the elapsed time. The response curve to training generally changes in shape as a function of time and training. Not only is there observed non-linearity but the shape of the non-linearity is itself changed by the process. In a common experience, a new physical skill may be quickly demonstrated (training by example) and then acquired at a rudimentary level by simple practice against a known objective. Basic proficiency may come easily but high level performance such as accuracy, while fundamentally incremental, generally takes much longer practice. Ultimate performance limits are reached slowly. Performance ‘trapping’ is common and requires a separate feedback process from that of the basic training.

A specific and new use of the emergent property of the data is the incorporation of a second feedback loop into a training process to avoid trapping in local areas of optimization. By separating training outcomes (exercises) it is possible to allow self-correction within the bounds of an exercise which may differ from the desired end state and thus separate training from initial assessment and the process for modification of the desired end stage. Rapid, precise and easily acquired data is preferable for this type of training and is enabling. Since the system being tested (neuro-musclulo-skeletal) is complex, it is beneficial to be able to direct training to specific components (sub systems) of the response.

As an example, in the learning of a complex physical skill such as a golf swing a level of overall performance may be learned through a single feedback loop and optimization however there may be no assurance that such optimization is global as opposed to local. A second optimization training process may be required to determine the possibility of further performance enhancement and may not be defined within the same measurement framework.

In settings involving re-training after, trauma or neurological damage for example, there is often an observed lag period before the generation of responses which may reflect underlying physical phenomena such as the regeneration or “rewiring” of nerve conduction channels or development of “alternate” neuromuscular pathways—all of which create complex response curves which differ in fundamental shape as they reflect multiple components which contribute to the overall responses observed.

After initial assessment, a first prescription for training is generated either manually by a trained practitioner or by an algorithm based on a training model. This “initial prescription” is a combination of the assessment and a set of outcomes (exercises) which may be measured as performed with immediate feedback to the subject in terms of the desired outcome of the specific exercises (not necessarily the desired end state or modification of the initially assessed state) for the purpose of allowing self-correction within the bounds of the exercise. This feedback loop provides a number of benefits including motivation, targeting (constraint) and the ability to separate this training stage from the initial assessment and process for modification of the desired end stage.

One or more embodiments of the present system do not require an explicit biomechanical model since an aspect of the invention is the restriction of performance to a defined task. Many such tasks may be utilized singly or in combination and it is a feature of the method that although constrained, the measured variables (e.g. force, displacement, velocity) are not predicted from the task definition. According to one or more embodiments of the present invention, analysis of the data obtained from a given performance need not be explicitly related to functional aspects of performance but rather may be carried out by correlation or pattern matching methods. The use of a specific task (Protocol) provides an implicit biomechanical frame of reference so that data between individuals or longitudinal (time) measurements on the same individual may be compared.

In practice of one or more embodiments of the invention, a subject or patient's interaction with the apparatus is mediated by an operator or therapist who typically but not necessarily provides additional guidance during the interaction. For example with respect to placement of measurement apparatus relative to the subject.

As a typical use is described, it is noted that the apparatus and method of its use is not restricted to a particular measurement or sensor type nor is it restricted to measurement and assessment of a particular task or class of subject. Specific examples of use are given for illustration of the general utility and not intended to be limiting.

In a particular instance of the apparatus, a single or multiplicity of sensors may be used simultaneously to gather data on more than one performance variable. This is illustrated in FIG. 1 where the term sensor refers to any of several single device nodes which may contain multiple transduction elements (such as an inertial measurement unit (IMU).

Thus in a particular instance of use, a sensor device may be a balance board which in turn contains several force transducers so that variations of force in an horizontal plane may be measured when a subject is standing on the sensor device 110 of FIG. 1.

The sensor device (balance board) 110 preferably pre-conditions data and produces digital data which are transmitted directly or through an internal radio link to a gateway device 120 which may be a laptop computer but is more typically a data buffer and relay that may act as a local hub for multiple sensors and a local display unit which provides feedback to the subject. This display unit is linked to the main apparatus but need not be directly linked to the local buffer. The display unit may provide performance guidance with respect to the specific task on which measurements are being made.

Within the category of balance assessment the following example is illustrative of the utility and functioning of system of FIG. 1. It is known that people with low back pain have an impaired ability to recover postural stability after internal perturbations induced by arm movement. Also, when compared with their age and gender matched pain-free controls, low back pain participants take a longer time to regain postural stability and typically require a greater number of postural adjustments during recovery.

Thus, expected outcome measures include time to postural stabilization and number of postural adjustments during postural recovery. For example, following bilateral rapid arm movement, patients with low back pain exhibit a longer time to return to pre-movement postural stabilization compared to age and gender matched controls. Therefore, low back pain subjects consistently took longer for postural recovery after voluntary arm movement. Additionally, the number of postural adjustments that occurs during the period between the onset of shoulder movement and postural recovery during the rapid arm movement has been shown to be significantly greater in low back pain patients compared to age and gender-matched controls

Consequently, the following protocol may be employed. As discussed above, implementation of the protocol may include displaying instructions to the operator at the client application 130. These instructions may include: Have the patient stand comfortably barefoot on the balance board; Ask the patients to close their eyes; Tell the patient that, “following a “beep”, they will have to rapidly flex both of their arms from their side to 60° flexion as fast as possible while maintaining their balance in the pre-set stance position. They will do this maneuver with their eyes closed for 2.5 seconds”; Ask the patients to close their eyes; In response to an auditory signal (given by the computer); subjects will have to rapidly flex their arms bilaterally at the shoulders to around 60° flexion as fast as possible while standing on their pre-set stance position; Encourage subjects to maintain equal weight bearing during the maneuver; Subjects will get 3 practice trials; When collecting data, patients will be asked to perform five individual trials; A 30 second rest will be provided between trials.

During performance of the protocol at least the following data are preferably collected: Weight; Force top right (FTR); Force top left (FTL); Force bottom right (FBR); Force bottom left (FBL). Again, in the present example, the sensor employed provides force measurements near the corners of the balance board. A simpler balance board providing only X-Y measurements may alternatively be employed.

Some additional information about the preferred performance of the protocol and data collection includes the following. An auditory warning preceded the trigger by a random period of 0.5 to 2 seconds. Three practice trials were provided before data collection. Data were collected at 100 Hz for 2.5 seconds, from 0.5 seconds before to 2 seconds after the auditory trigger for each trial The time of shoulder movement onset and peak, duration of movement and peak range of movement were identified using the movement trace recorded by the motion analysis system

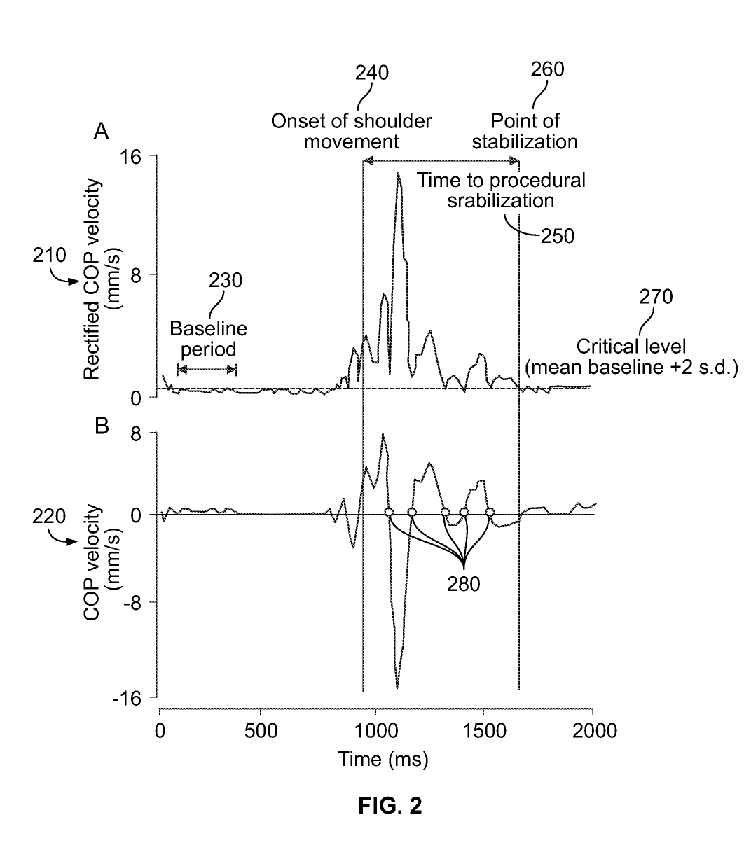

FIG. 2 illustrates the rectified COP velocity 210 and the actual COP velocity 220. FIG. 2 also illustrates the baseline period 230, the onset of shoulder movement 240, the time to procedural stabilization 250, the point of stabilization 260, and the critical level 270. Additionally, the circles 280 represent the times at which the COPVap crossed zero. Five crossings are identified in FIG. 2. From the data shown in FIG. 2, a number of important values may be determined.

First, the COP Excursion (COPap) which is the distance between the Maximum AP (Anterior/Posterior) position and the minimum AP position. In FIG. 2 the variables in the AP dimension were analyzed as postural perturbation induced by the arm movement occurred primarily in the sagittal plane.

Second, the COP Velocity (COPVap) which is calculated from the instantaneous position of COP during the trial. In FIG. 2, the variables in the AP dimension were analyzed as postural perturbation induced by the arm movement occurred primarily in the sagittal plane.

Third, the Time to postural stabilization which is the time taken from the COPap velocity to return to a pre-perturbation level was calculated. It was calculated as the time for the rectified COPVap trace to return to a level consistent with the baseline (mean COPap from 100 ms to 400 ms before onset of shoulder movement plus 2 SDs or Standard Devisations), and remain below this velocity for 30 ms following shoulder movement.

Fourth, the Number of adjustments which is the number of adjustments that were recorded as the number of times the COPVap crossed zero (which represents major direction change of the COPap trajectory) in the period from shoulder movement onset until the time to stabilization using the plot of un-rectified COPVap against time.

In one embodiment of the present invention, data acquisition may be done in local loop mode wherein the test protocols guiding performance are downloaded to the local gateway (laptop) and the data stored locally for subsequent transmission to the main server.

One feature of the system is that performance measurement is related to specific tasks (Protocols) that define and constrain performance and thus the subject interface (typically a screen display of graphical information) may be driven directly from the local buffer or from the main processer/server.

The main server or web application 140 receives data from the sensors through the gateway 120 and is linked to one or more data repositories (such as data bases). It is also linked to a ‘task library’ which comprises a number of specific performance tasks which are used to guide/drive the test process and which are specified by an operator or therapist. The test library (instructions) is a component of the method as it provides biomechanical constraints for data interpretation. One or more display devices may be driven by the main server for the purposes of display of data acquisition and interpretation. These displays may be linked to the server 140 through the internet and browser driven so that the display is independent of device specific constraints.

This allows a therapist to have local access in ‘real time’ for feedback as well as ‘off line’. Additionally the main server or a complex of servers provides the computational resources necessary for data analysis (including analysis in reference to other performance data either from population data bases or longitudinal (time) subject data or both. Such data may be combined for analysis with other data sets such as medical record or other information not necessarily related to specific task performance. Reports generated may be transmitted to other relevant systems across standard application interfaces.

In one embodiment of the system of FIG. 1, a sensor system comprises a balance board (e.g. Nintendo Wii); this sensor device includes force transducers and integrated on-board signal processing, digitization and a Bluetooth radio link. The radio signal may be received by a compatible Bluetooth radio device which, in turn may utilize a standard USB port on a conventional laptop computer device. Such a Bluetooth-equipped laptop may function as a gateway for the purposes of the present system and preferably has an integrated display suitable for presentation of test performance data and instructions when driven by suitable software. The software may be configured so as to provide graphic confirmation of test performance using displays of data or symbolic icons or text as desired. Any sensor-gateway link, wired or wireless may be used in the practice of the present system.

In another embodiment of the system of FIG. 1, a sensor system may comprise one or more of a grip-strength device or a force measurement device of a nature and type commonly utilized in physical training and assessment practice. Such devices are manufactured items in commerce and may easily be fitted with transducers and transmitters as desired.

In another embodiment of the system of FIG. 1, a sensor system may comprise one or more of an accelerometer or an IMU (such as a device combining an accelerometer, a gyroscopic sensor and a magnetometer) all of which are in common use in current biomechanical measurement practice. Similarly, positional inputs may be measured by video-based sensor systems. Novel measurement devices may be used without limitation in the practice of the invention.

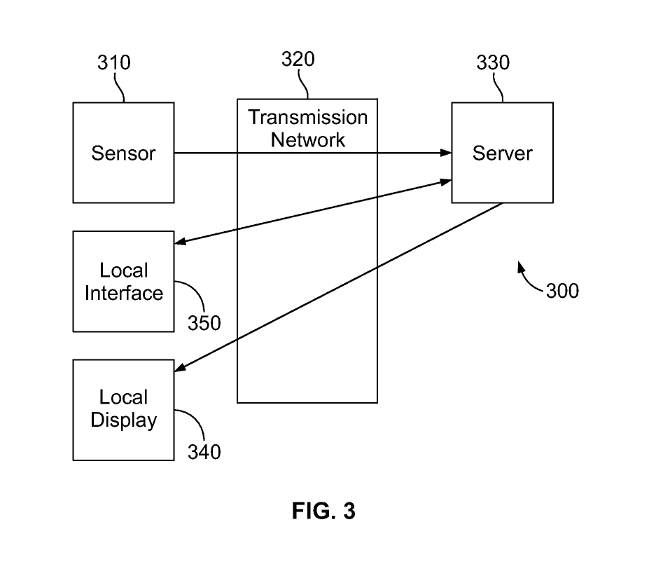

FIG. 3 illustrates an alternative embodiment of the task-associated motion analysis system 300. The task-associated motion analysis system 300 includes one or more sensors 310, a transmission network 320, a server 330, a local display 340, and a local interface 350. The sensor 310 may be any of the sensors discussed above. In the embodiment of FIG. 3, the data from the sensor passes through a transmission network 320 to the server 330. For example, the sensor 310 may be a web-enabled device that may be configured for plug-and-play integration into a standard web connection. When activated, the sensor 310 may transmit its data directly to a remote server 330 through the transmission network of the internet 320. Additionally, the communications from the sensor 310 to the server 330 may be secured through the use of passwords and/or encryption.

Once the data is received at the server 330, it may be processed as described herein. A graphical display may also be sent from the server to a local display 340 through the transmission network 320 such as the internet. The local display 340 may for example be a screen on a device that may provide a display viewable by the operator and/or patient.

Additionally, the server 330 may communicate with the local interface 350. The local interface may also control the sensor 310. For example, the operator may use the local interface 350 to initiate a protocol for a sensor. Once the sensor 310 records the data and transmits it to the server 330, the server 330 may analyze the data and transmit a command, instruction, or warning to the local interface. For example, the server 330 may request that the protocol be repeated or may identify a different protocol to be employed, or may provide another instruction to the operator with regard to how to position the patient or to provide instructions to the client. Additionally, based on an analysis of the data, the server 330 may send questions and/or instructions to the local interface for the operator to perform. For example, one such question might be “Have you noticed a decline in your ability to do [a specific task]” or “How long have you been experiencing problems doing [a specific task]” or “Do you feel discomfort/strain in any of your knee, hip, ankle, calf, quad, hamstring, gluteus, lower back, stomach? If so, please specify”. The interface 350 also includes an input that allows the operator to indicate the patient's selection. Such an input may include a keyboard, touchscreen, or buttons, for example.

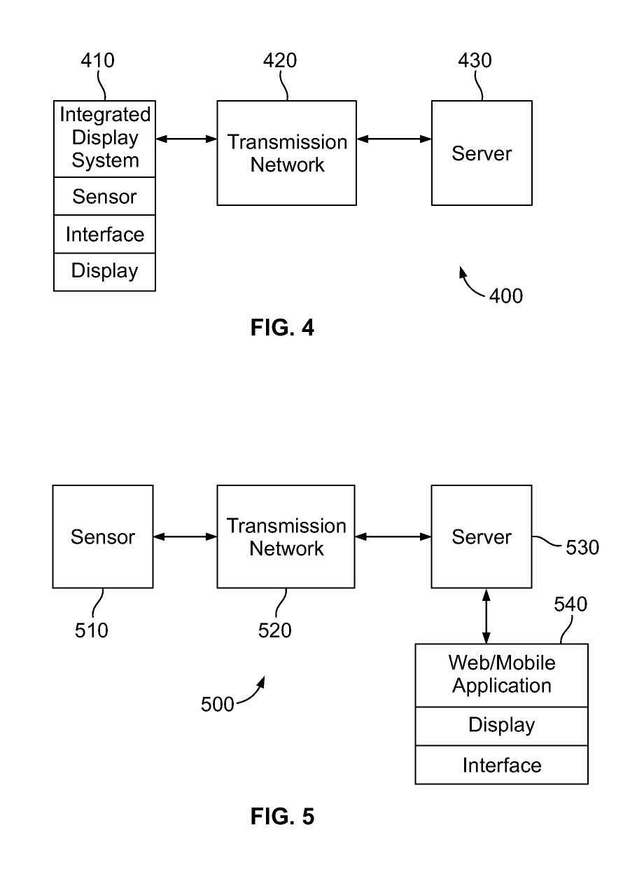

FIG. 4 illustrates an additional alternative embodiment of the task-associated motion analysis system 400. The task-associated motion analysis system 400 includes an integrated display system 410, a transmission network 420, a server 430. The embodiment of FIG. 4 is similar to that of FIG. 3, but the sensor 310, local interface 350, and local display 340 of FIG. 3 are replaced with an integrated display system 410. The integrated display system 410 is preferably a single system wherein the system, interface, and display components are directly connected through a wired or wireless link.

In operation, the interface of the integrated display system 410 is used to activate the sensor which then records data and transmits it to the server 430 through the transmission network 420. Once the server processes the data, display data may be transmitted back through the transmission network 420 to the integrated display system 410 for display on the integrated display system's display. Additionally, the server 430 may send queries, interact with, and receive data and/or commands from the integrated display system's interface.

FIG. 5 illustrates another alternative embodiment of the task-associated motion analysis system 500. The task-associated motion analysis system 500 includes one or more sensors 510, a transmission network 520, a server 530, and a web/mobile application 540. The embodiment of FIG. 5 is similar to that of FIG. 3, but the sensor 510 is in bi-directional communication with the server 530 through the transmission network 520. Additionally, the web/mobile application 540 is also in bi-directional communication with the server 530. For example, the web/mobile application 540 may communicate with the server through the cellular phone system or through the internet.

In operation, an account may be created at the server 530 which may be password and/or encryption protected. The account may then be associated with one or more sensors 510 and the web/mobile application 540 which may be hosted, for example, on smartphone or a tablet computer such as the iPad®. Additionally, the server 530 may establish a premises account associating one or more sensors with one or more web/mobile applications.

In operation, once the sensor and web/mobile application have been associated at the server, the operator may position the patient with regard to the sensor and then initiate the testing protocol using a testing initiation command entered through the interface of the web/mobile application. The testing initiation command may then be transmitted to the server 530 which may identify the associated sensor and then send a command to the sensor 510 through the transmission network 520 in order to activate the sensor. When more than one sensor 510 is present, the web/mobile application 540 includes on its interface a selector for selecting the desired sensor to initiate.

Turning now to specific measurements, in general there are multiple options for measurement of position, force and rate of change of the elements of the musculo-skeletal system and inputs from such measurement subsystems may be utilized in the practice of the present system

A novel and unexpected result of the present system is that assessments of performance, when associated with defined tasks, are informative of the functional state of the musculo-skeletal system without explicit reference to the specific task. Some performance outputs reflect the results of fundamental functional processes which are convoluted with the signals of a defined task performance. As specific, but not limiting examples:

First, the force vs time (frequency) components at higher (for example, greater than 2 Hz) frequencies are informative as to involuntary processes including pathological tremors and loss of strength.

Second, the displacement around the center of mass (measured by changes in the center of balance or by an IMU placed near the center of mass or by video analysis of upper body motion) is informative as to the ability to maintain balance.

Third, the rise, hold and return to baseline in strength tests (grip or muscle strength) are all significantly informative of general fitness as well as the condition of the specific musculo-skeletal elements invoked in the specific task measured

Accordingly, a novel utility of the present system is the characterization of aspects of musculoskeletal health such as balance and posture control, muscular strength and endurance and movement kinematics by using suites of specific defined tasks. Quantitative measurement of mechanical outputs associated with the defined tasks allows comparison (correlative or explicit) with population results and or time series from the same subject. These comparisons create profiles within areas of performance (such as frequency domains or differential motion) that are associated with musculoskeletal health.

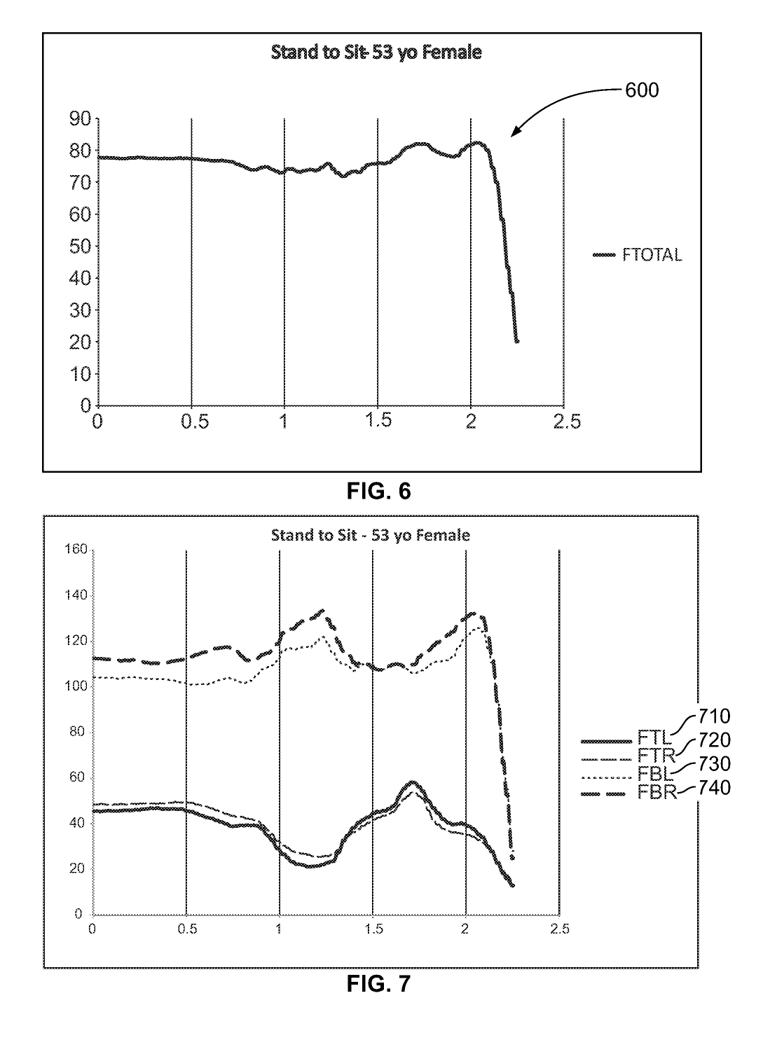

FIGS. 6-18 below are examples which are illustrative but not limiting which display force vs time charts for a number of specific tasks as performed by several different subjects. Attention is drawn to specific qualitative and semi-quantitiative features of these graphs to illustrate how various analytical methods may be applied with respect to both general and specific performance features.

FIG. 6 shows a graph 600 of the total force (Ftotal) observed using a force plate for a 53 year old female subject performing a stand-to-sit task.

FIG. 7 shows graphs of the Force top left (FTL) 710, Force top right (FTR) 720, Force bottom left (FBL) 730, and Force bottom right (FBR) 740 for the same subject as in FIG. 6.

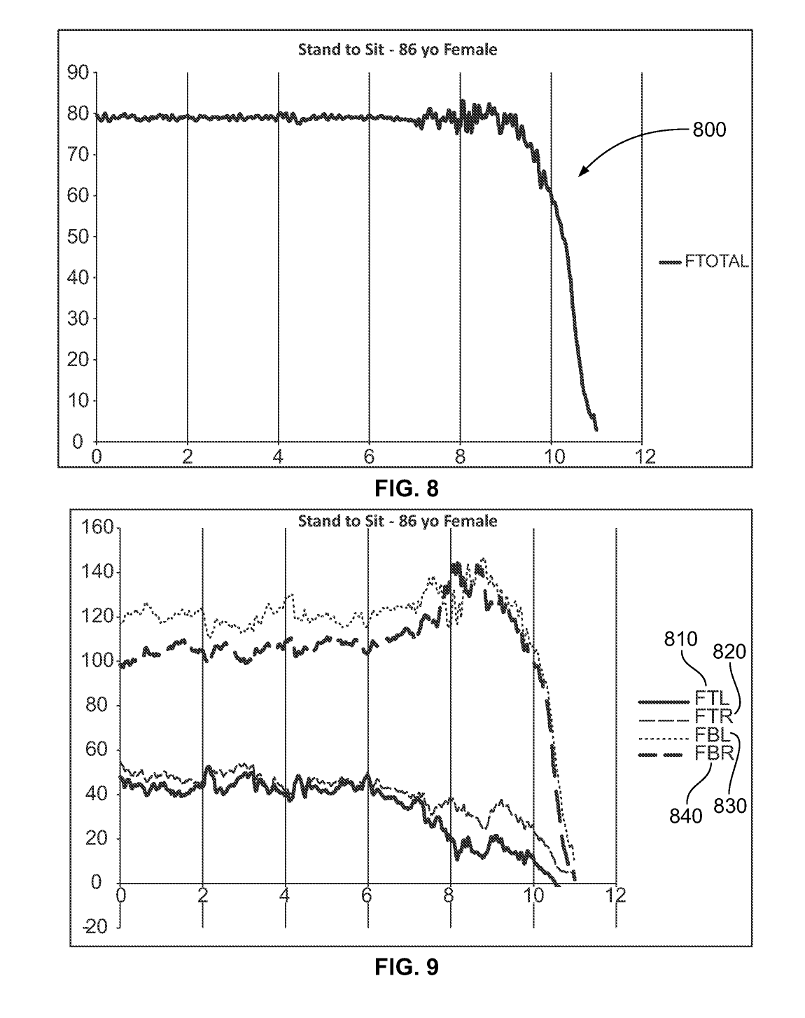

FIG. 8 shows a graph 800 of the total force (Ftotal) observed using a force plate for a 86 year old female subject performing a stand-to-sit task.

FIG. 9 shows graphs of the Force top left (FTL) 910, Force top right (FTR) 920, Force bottom left (FBL) 930, and Force bottom right (FBR) 940 for the same subject as in FIG. 8.

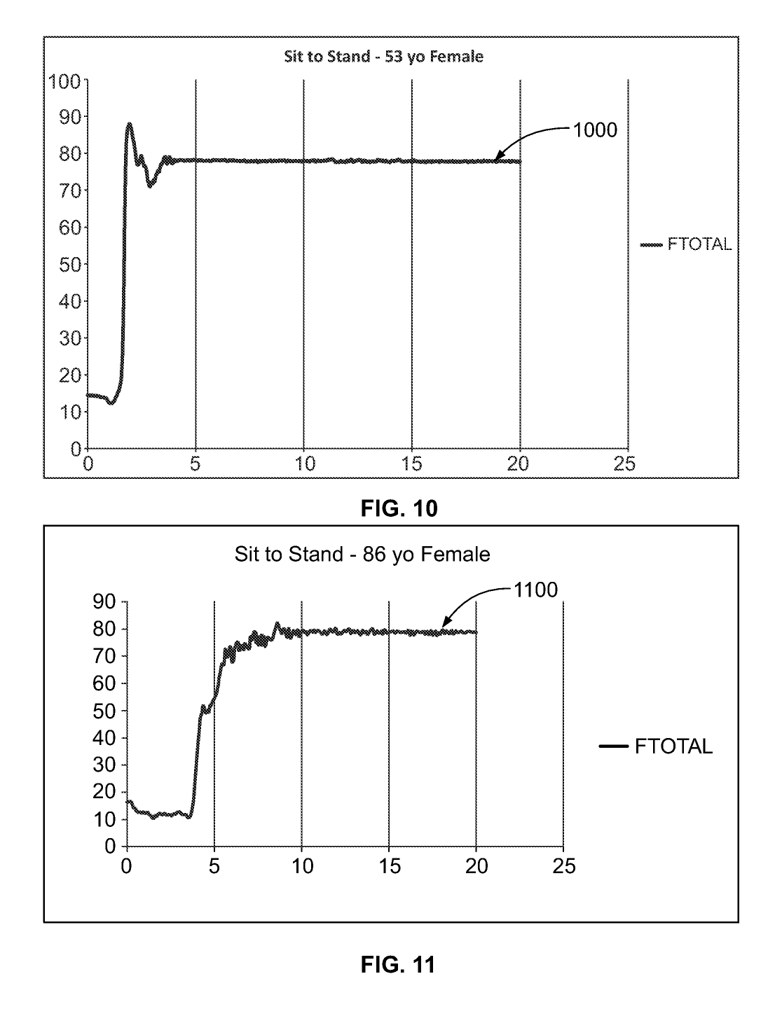

FIG. 10 shows a graph 1000 of the total force (Ftotal) observed using a force plate for a 53 year old female subject performing a sit-to-stand task.

FIG. 11 shows a graph 1100 of the total force (Ftotal) observed using a force plate for a 86 year old female subject performing the same sit-to-stand task as in FIG. 10.

With regard to FIG. 6-7, the 53 year old female is reasonably fit and consequently the curves shown are relatively illustrative of the performance expected from a reasonably fit individual of that age and gender. In comparison, FIGS. 8-9 show an 86 year old female with significant postural instability. Note the expansion of the time axis and increase in high frequency components FIG. 8-9.

FIGS. 10 and 11 show a side by side comparison of the performance of the relatively stable 53 year old female and the significantly unstable 86 year old female. These figures show the change in nature of task performance with respect to both shape of curve and time to complete. The shape of curve may be analyzed by using polynomial fit or other standard data reduction methods in order to allow quantitative comparison and it should be noted that in transitioning from one curve to the other (specifically the 53 year old subject's performance curve to that of the 86 year old) there is a basic difference in the functional description that may generate an unambiguous indication of change in condition. As an example, calculation of the first derivative of a polynomial function fitted to the performance curve would indicate transition from an ‘overshoot’ to a gradual approach as shown explicitly in the data displayed.

Similarly, comparison of the data sets in FIGS. 10 and 11 shows significantly larger excursions at high (greater than 5 Hz) frequencies and thus a power spectrum analysis of such data may provide objective measure of ‘fitness’ or an indication of the progress of aging.

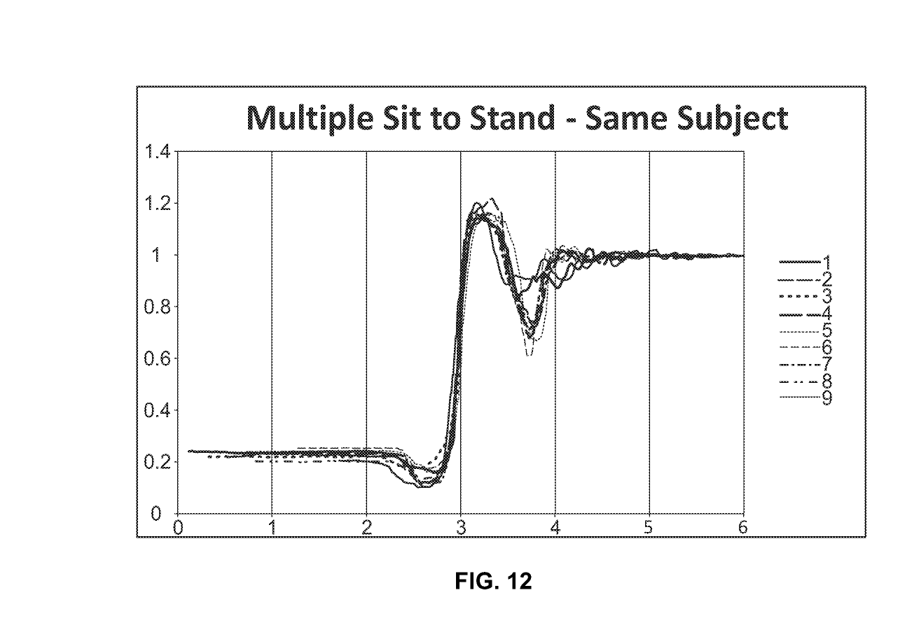

FIG. 12 illustrates multiple performances of the sit-to-stand task by a middle-aged, fit male. As shown in FIG. 12, the force vs time data associated with a standard task is consistent over multiple repeats; the effect of fatigue after multiple trials (short of exhaustion and task failure) is progressive and results in dispersion of the data.

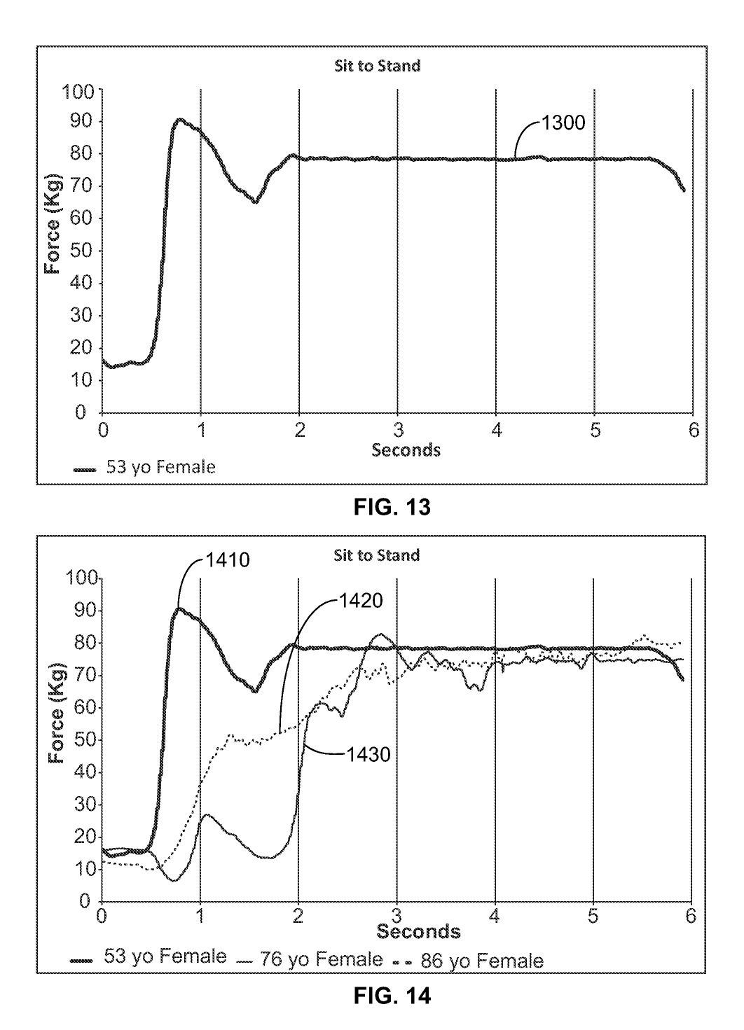

FIG. 13 shows graph 1300 of the total force (Ftotal) observed using a force plate for a 53 year old female subject performing a sit-to-stand task.

FIG. 14 shows graphs of the total force (Ftotal) observed using a force plate for a 53 year old female subject 1410, 76 year old female subject 1420, and 86 year old female subject 1430 performing a sit-to-stand task.

FIGS. 13 and 14 represent further illustrations of inter-subject differences that reflect aging/fitness (53, 76, 86 year old individuals on a single trial of sit-to-stand task). As shown in FIG. 14, the sit-to-stand task shows one or more of the following with increasing age: additional time to max force, additional high frequency components with increasing age, takes longer to reach steady state, more low frequency amplitude oscillations during the task, etc.

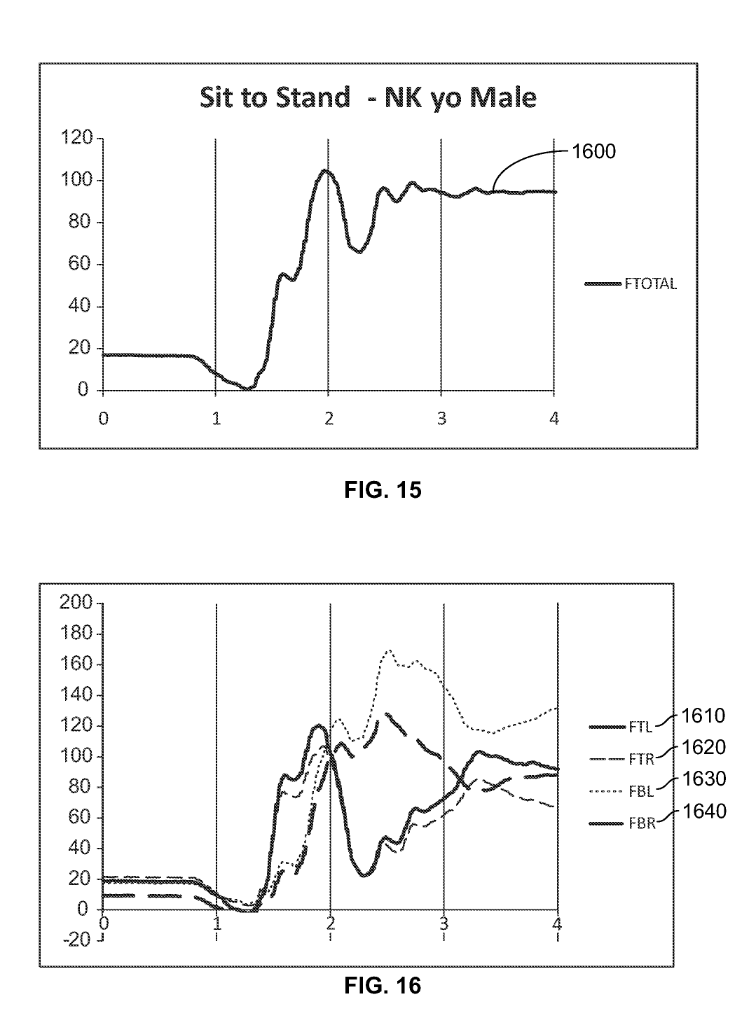

FIGS. 15-17 illustrate sit-to-stand data for a fit middle-aged male with significant left-right asymmetry.

FIG. 15 shows graph 1500 of the total force (Ftotal) observed using a force plate for the middle-aged male subject performing a sit-to-stand task.

FIG. 16 shows graphs of the Force top left (FTL) 1610, Force top right (FTR) 1620, Force bottom left (FBL) 1630, and Force bottom right (FBR) 1640 for the same subject as in FIG. 15,

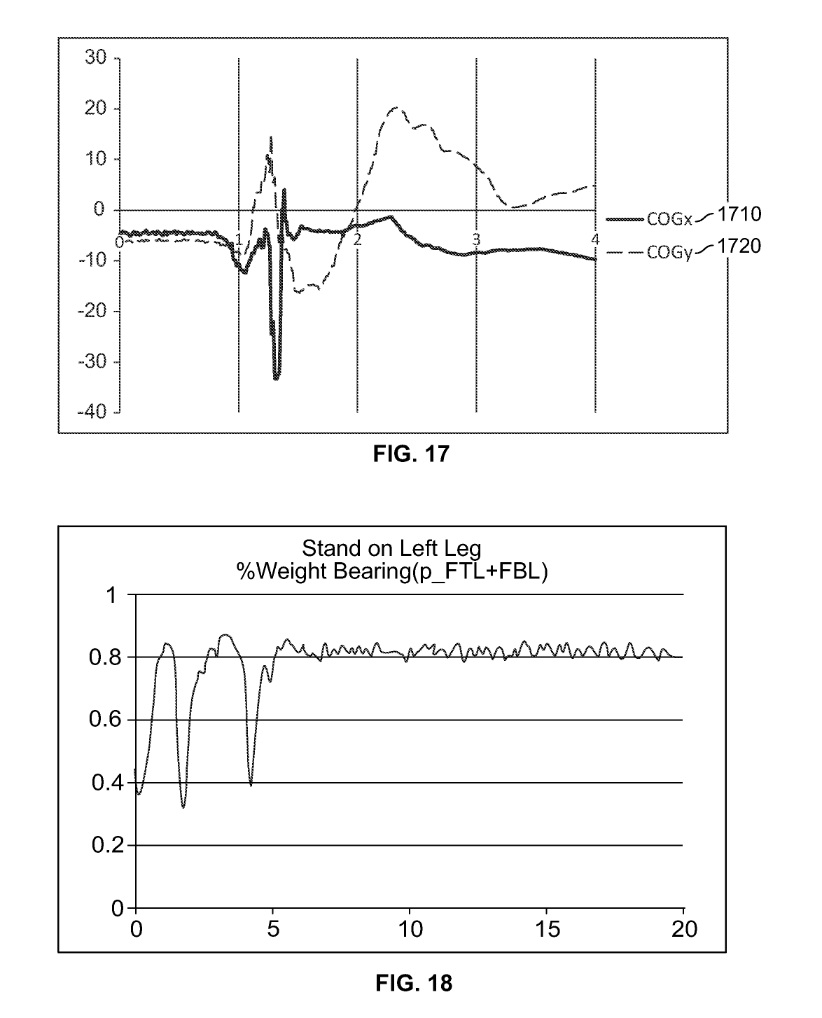

FIG. 17 shows graphs of the center of gravity in the x direction 1710 and the center of gravity in the y direction 1720 for the same subject as in FIG. 15.

As shown in FIGS. 15-17, the significant left-right asymmetry in performance as may be easily and quantitatively assessed from FIGS. 16 and 17 which display front and back left and right quadrants in FIG. 16 and Center of Gravity (left and right) in FIG. 17. Such asymmetry may be indicative of developing joint or muscle problems. More specifically, for example, the asymmetry may be seen in FIG. 17 because the two COG graphs are not symmetric.

One or more embodiments of the present system and method of its use use baseline or standard data to populate data bases for comparison and therefore an emergent property of the system is that it becomes increasingly useful as the data bases increase in size and segmentation by relevant (demographic or medical) subcategories becomes possible. This emergent property provides real time access to comparative data that is highly desirable.

As data bases grow in size, the effectiveness of the present system increases through the ability to identify performance bounds and correlate them with specific demographic or medical information. For example, a time series of strength measurements on a single subject is observational and of limited diagnostic utility; comparison against normative data may readily identify ‘outliers’ which merit further attention. For greater clarity, the same situation exists in relation to chemical analyses of (for example) blood where analyte concentrations require comparison to ‘reference values’ to be generally useful and where groups of analytes co-vary in a manner analogous to specific attributes of performance.

A consequence of the analysis of specific attributes of performance as taught by the present system is that therapeutic recommendations may follow from diagnostic analysis. Specifically, and by way of a non-limiting example, if an analysis of loss of ability to maintain balance indicated that this is correlated with muscular strength loss, a recommendation would be muscle-strengthening exercises. Were the same functional loss related to a neurological deficit these exercises would not be appropriate. The ability to quantitatively assess and track progress in therapy is important both as to assessment of efficacy and improvement of motivation.

The analysis and recommendations made are emergent properties of the system that result from its use with increasing population size and diversity. Analytical methods for dealing with large data sets are well known in present art as are techniques for presentation of such results.

Below are some additional examples of one or more embodiments of the present system:

### Example 1

A subject stands on a horizontal ‘balance board’ (a plate with a number of load sensors so that shifts in force in the X-Y plane of the balance board may be detected; the time constant of the device should (preferably) allow data to be acquired at a frequency greater than 20 Hz. Once the subject is in position, tasks such as ‘maintain static position for (say) 1 minute’ or ‘close your eyes and maintain static position for (say) 1 minute’ or ‘with your eyes closed, lift your arms to horizontal in front of you and hold this position for (say) 1 minute’ are performed and data from the load sensors is captured via a standard (digitizing) interface and transmitted wirelessly to a gateway.

The data stream may be immediately displayed to both the subject and the test operator if feedback is desired and simultaneously sent through a network to a computer or computers where it is entered into a database and then analyzed by comparison with performance data obtained through carrying out the same task.

Such analysis may compare frequency distribution of ‘excursions around mean values’ or aperiodic features such as lateral drift and requires for its interpretation comparison to other subjects, preferably with known demographic or other attributes. Further, the present system becomes increasing effective with the development and use of data bases which record measurements as data sets associated with specific task performance and demographic data.

In another embodiment, the analysis may compare the data received with an idealized curve representing the desired characteristics for performance of the task. The idealized curve may be scaled based on one or more of the gender, weight, and age of the patient.

### Example 2

A balance board, as in the previous example is wirelessly linked to a computer hosting data acquisition software and a user-guiding test protocol which defines specific tasks. On a signal or the instruction of a tester (therapist) a subject attempts a sit-to-stand motion sequence starting with both feet on the balance board and ending with the subject in a stable standing position. (alternately a stand-to-sit task may be specified). The data set for the test may be defined either from an arbitrary start time or alternately by a defined time series counting back from the end of the test sequence. The latter avoids start transients and is generally more easily determined in a clinical setting.

Several repeats of the defined task (sit-to-stand) may be performed. Immediate feedback from the sensor system is optional and generally helpful to both the patient and therapist since it provides verification of task performance.

Performance data sets are tagged with identifying information specific to the subject and session and transmitted through the (secure) network to a data repository and data analysis where using methods generally known in mathematics (signal extraction, averaging and statistical processing) relevant performance information may be extracted and compared to both population data and time series information for the particular subject. The ability to do quantitative comparisons of specific task performance data across defined populations as well as over time is a feature of the present system. It should be noted that such comparisons do not require an explicit biomechanical model of the task being performed. For example, in a sit-to-stand task, several features of the force-vs-time curve reflect inter-subject variations. With reference to the figures above and without limiting generality, features of the force-time graphs may be visually distinguished and provide specific opportunities to characterize and compare task performance. Examples within this set of graphs include (without limitation): duration of rise, overshoot and ‘settling’, low amplitude oscillations at relatively high frequency, and lateral asymmetry.

Other features of the graph may be identified and data may be analyzed using standard methods or algorithms developed for feature extraction. Note that, in general, data sets may be scaled or normalized for the purpose of comparison between subjects or repetitions by the same subject.

It should also be noted that such performance-specific data are consistent for a given subject within a small number of repeats session. The effects of fatigue or training are rapidly manifested as shown in FIG. 12 above.

The general shape of the curve changes with physical condition in ways that reflect loss of ability to perform a specific task which may be a proxy for more general functional ability

As an example, compare FIGS. 6-9 which illustrate that the same task, performed by different individuals may produce significantly different performance data. In this specific example the two subjects performed the same specified task but had different physical abilities correlated to their ages.

Such correlations may be established experimentally thus the inclusion of a data base of performances together with relevant demographic information is a desirable and distinguishing feature of the present system.

A general relationship exists between specific task performance and functional ability since specific task performance invokes multiple sub-activities; this allows performance comparison across populations by demographic or other segmentation criteria.

Data reduction (for example polynomial curve fitting and differentiation) may provide an indicator of fitness or strength. In comparison to a young and vigorous performance as shown in FIGS. 6-7 there is a fundamental shape change in the force-vs-time graph for an elderly and somewhat infirm subject as shown in FIGS. 8-9. Specifically, FIGS. 6-7 have a pronounced overshoot result of ‘bounce’ while the elderly subject's performance of the same task indicates both a slower time to rise and a fundamentally different force-vs-time curve. Frequency analysis of the detailed data may reveal strength or neuromuscular control issues, and lateral asymmetry in force-vs-time curves may be indicative of injury or disease.

### Example 3

In a practical (clinical) setting it is desirable to include information from a patient's musculo-skeletal record in their medical record and to be able to record and annotate management and billing records. It is clear to a person skilled in computational management of data that such data may readily be passed back to or from the system described above and that such a system may be configured to the requirements (privacy and security) of medical records management. Accordingly in an embodiment of the present invention, the system permits access to and from medical and management data systems for reporting purposes and may be integrated with such systems.

### Example 4

The performance of the present system increases with its being used in conjunction with testing protocols which are consistent as to task performance requirements and designed to provide implicit biomechanical models. Specific tasks invoke constrained performance by subsystems of the overall musculoskeletal system and, in addition, may interrogate or reflect the control of these components by the nervous system. Accordingly it is anticipated that using suitable tasks it will be possible to probe functional aspects of control of the musculoskeletal system by the nervous system without explicit testing.

As an example, the high (greater than 1 Hz and less than 100 Hz) frequency components of force vs time or displacement vs time data sets contain information about involuntary tremors such as are seen with Parkinson's disease or ‘intentional tremor’ and thus may have utility in diagnosis and monitoring such disorders.

### Example 5

Measurement of strength and endurance is common practice in physical assessment and such tests, generally done with explicit force measurement devices, are generally recorded as peak (maximum) force and duration achieved, sometimes with multiple repetitions. Detailed data on the approach to peak and higher frequency components of the response during the ‘hold’ phase of such tests may readily be obtained with available transducers including load cells and accelerometers. Such data may then be analyzed with reference to normative data from populations using the basic methodology and system of the present system. Changes in the frequency distribution of applied force vs time within a defined task are diagnostic of condition and, if observed in a longitudinal test series for a given subject may be diagnostic of pathological changes or training benefits. Such changes may be observed in explicit strength and endurance testing and also within complex functional performance such as maintenance of balance.

FIG. 18 illustrates a force-time graph showing a subject establishing and maintaining a one-legged stance. As shown in FIG. 18, the approach to steady state illustrates several cycles of attempt and correction. Comparison with demographically and functionally relevant data obtained from the same stereotypical performance, increases the ability to interpret such data in terms of the likelihood that the subject has or will develop functional balance problems. By comparison with such data bases within the present system and method, such useful interpretations are provided.

### Example 6

Within an established population, a specific task set may be used to establish a data base that reflects changes as the result of training (repetitive exercises). By measuring the change of performance vs time of multiple subjects it is possible to derive measures which reflect the effectiveness of a trainer or therapist. This inversion of the functional utility of the present invention does not rely on explicit performance of either the trainer or the trainee.

Although only a few exemplary embodiments of this invention have been described above, those skilled in the art will readily appreciate that many other equivalents are possible without materially departing from the novel teachings and advantages presented herein. Accordingly, all such modifications are intended to be included within the scope of this invention.

Thus, one or more embodiments of the systems and methods described herein comprises several elements which in combination may measure attributes of task-associated motion which yield information relevant to assessing and improving performance. Multiple embodiments and applications over wide fields of use both in diagnostic and therapeutic contexts have been presented. Specific references to physical training and therapy are not meant to restrict the application of the invention. Assessment of task-specific performance may not require an explicit biomechanical model. One or more embodiments of the present system further address a basic limitation of assessment and training systems that arises from their self-referential nature.

In addition to the regions or aspects of the curves identified as relevant above, any of the following regions or aspects of the curves related to the performance of a task may be used for evaluation alone or in conjunction with others: A) Time from start of recording to initiating of action, B) Smoothness of rise (or slope), C) Presence of “Hitch” in rise, D) Time to rise from initiation to peak, E) Overshoot percentage from steady state, F) Speed of oscillation near steady state, G) Amplitude of oscillation at steady state, H) Time to steady state, I) Number of zero passes, and J) Concave-ness of curve.

For example, if, when analyzing a specific task-associated curve, the time from rise initiation to peak is greater than a certain threshold, then the slowness of rise correlates with a specific condition such as one or more of the following: balance issues, musculoskeletal weakness, arthritis, and joint problem. In one embodiment, the threshold may be 2 seconds, in another embodiment, the threshold may be 1.5 or three seconds.”

Additionally, the curve for comparison with a patient's data may be an idealized curve, a composite of numerous healthy people, or may be broken out by age/sex cohort for comparison. Additionally, the thresholds identified above may be adaptive for age/sex cohort comparison.

Also, the present system may provide an automated training or therapy recommendation. For example, if subject data is found to have exceeded a threshold in as described above, and was identified as being correlated with a specific condition, then specific training or therapy recommendations are suggested For example, identification of balance issue yields a recommendation of 5 mins/3×per day of standing on a balance improving device. Other options include specific exercise to strengthen muscles identified as weak and may include surgery and/or medication.

Additionally, the testing protocols may include any of several options. For example, the testing protocol may call for performing more than a single measurement to provide data for analysis. For example, the protocol may direct the patient to perform the same measurement type multiple times and average/interpolate results for analysis. Alternatively, the protocol may: direct the patient to perform two or more different type measurements in a set pattern, direct the patient to perform two or more different type measurements in an adaptive pattern (wherein the second or subsequent measurements depend on the results of preceding measurements), when two or more different types of measurements are performed, present/analyze results separately; and/or when two or more different types of measurements are performed, combine the results of both for analysis.

Additionally, as mentioned above, the central server may allow repository linking. For example, test results for a specific patient may be linked with previous test results, and/or medical and billing information for the client. The availability of the previously stored test results may allow the system to provide tending and/or tracking of performance information for the patient. Additionally, the medical information may include information such as surgeries or medication so that their impact may be considered when analyzing performance.

Also, as mentioned above, the present system may be implemented to provide a second feedback loop or a multi-modality training performance verification. For example, two or more training modalities may be employed—and employed multiple times over an extended time interval. The use of multiple modalities provides a better indicator of overall performance by testing in multiple fashions. For example, the first test may indicate that a first problem is present and that a first therapy, such as flexibility training is desirable. The second, different test modality may confirm this or at least may not contradict this. Flexibility training may then be performed for a predetermined time and the patient re-measured with both modalities. The first modality may indicate an improvement, but the second modality may not. This indicates than an alternative, replacement, and/or adjusted therapy is needed, such as adding resistance training in addition to flexibility training, for example.

Also, one or more embodiment of the present system may be integrated with other systems commonly used in the practice of medicine, such as accounting systems, insurance management systems, reporting of recommendations, therapy, and/or results to the patient's primary car physician, and reporting and/or website access to results by the patient over the internet.

Further, an embodiment of the present system may allow the evaluation of the therapists using the system. For example, the patient data may be statistically analyzed on a therapist-by-therapist basis in order to track and quantify improvements in patients for a specific therapist. Therapists of greater or lesser overall average effectiveness may be identified as well as therapists that perform better or worse with certain age cohorts, genders, or conditions. The system may offer or provide a referral to additional training/services if improvement of patients for a specific therapist is less than what is seen for other therapists.

Also, an embodiment of the present system may allow evaluation of the effectiveness of a therapy across a population generally or broken down into cohorts by age, gender, and/or condition. For example, it may be found that when ‘time to rise from initiation to peak’ is too large, the most effective therapy varies on cohort factors. For example the most effective training may be resistance training for those over 80 and flexibility training for those under 50.

While particular elements, embodiments, and applications of the present invention have been shown and described, it is understood that the invention is not limited thereto because modifications may be made by those skilled in the art, particularly in light of the foregoing teaching. It is therefore contemplated by the appended claims to cover such modifications and incorporate those features which come within the spirit and scope of the invention.

1. A system for task-associated motion analysis.
